(19) United States
(12) Patent Application Publication
(10) Pub. No.: US 2026/0158186 A1
(43) Pub. Date: Jun. 11, 2026

(54) OPTICAL DEVICE FOR DISINFECTING UPPER AIR LAYERS IN A ROOM

(71) Applicant: OSRAM GmbH, Muenchen, DE (US)

(72) Inventor: Ulrich Hartwig, Berlin (DE)

(21) Appl. No.: 18/706,689

(22) PCT Filed: Sep. 13, 2022
(86) PCT No.: PCT/EP2022/075386

§ 371 Date: May 1, 2024

(30) Foreign Application Priority Data
Nov. 4, 2021 (DE) 102021212448.8

(51) Int. Cl. A61L 9/20

(52) U.S. Cl. CPC A61L 9/20; A61L 2209/12

(57) ABSTRACT

In an embodiment an optical device includes a light source unit having a light source and a light-emitting surface, which defines a light source plane and is configured to emit radiation in a UV wavelength range, wherein the light-emitting surface is configured to emit the radiation from the light source unit in an angular range relative to a main radiation direction, which is perpendicular with respect to the light source plane, a reflector, which is arranged at a predetermined distance from the light-emitting surface in the main radiation direction, and which is configured to receive the radiation emitted by the light-emitting surface and to reflect it at least in a direction opposite to the main radiation direction, and a blocking element configured to absorb or reflect the radiation, which, due to its angle of radiation, passes from the light-emitting surface beyond an edge of the free form of the reflector.

### CROSS-REFERENCE TO RELATED APPLICATIONS

This patent application is a national phase filing under section 371 of PCT/EP 2022/075386, filed Sep. 13, 2022, which claims the priority of German patent application 10 2021 212 448.8, filed Nov. 4, 2021, each of which is incorporated herein by reference in its entirety.

### TECHNICAL FIELD

The present invention relates to an optical device for the disinfection of upper air layers in a room which is used, for example, by persons, in particular also to prevent infection with pathogens.

### BACKGROUND

Optical devices for disinfecting air, especially in enclosed spaces, have been recently increasingly used, particularly in the wake of the SARS-COV-2 coronavirus pandemic. Such devices generally use ultraviolet radiation, in particular UV-C radiation, to inactivate or kill germs or pathogens such as bacteria, bacterial spores, viruses or viroids, fungi, fungal spores or algae etc. from the room air.

In the case of UV-C wall-mounted devices or mobile UV-C devices frequently used for this purpose, air can be extracted from the corresponding rooms, exposed to UV-C radiation during treatment and then fed back into the corresponding room. A wavelength range corresponding to UV-C radiation ranges from 100 nm to 280 nm. Other wavelength ranges such as UV-A or UV-B radiation are not excluded. For example, low-pressure mercury vapor lamps can be used that emit radiation or light with a wavelength of 254 nm, which is used for virus inactivation, for example, as the virus nucleic acid is attacked in this case. After a large number of cycles, this treatment can reduce the germ load in the rooms concerned by more than 99 %.

Conventionally, mercury medium-pressure lamps or pulsed xenon arc lamps were also used in UV-C germicidal devices. Current devices mostly use low-pressure UVC lamps. More recently, efforts have also been made to use UV-C LEDs.

When using such devices, however, questions of radiation protection must always be considered, as the UV-C radiation released can also have a very damaging effect on people's eyes and skin etc. if they are exposed. Special measures are therefore generally required for the aforementioned wall-mounted appliances to ensure that the UV-C radiation does not escape from the interior of the appliance, such as angled inlet and outlet openings or slats.

Another type of germicidal UV-C device currently used in the United States is the so-called Upper Air or Upper Room GUV Device (GUV: germicidal UV). Such devices are recommended for rooms with a ceiling height of at least 10 feet (equivalent to approx. 3.048 m). The devices for UV-C disinfection installed at a height of e.g. approx. 2.1 m (corresponding to 7 feet) are installed in such a way that they only disinfect the air above their own height. They emit the UV-C radiation directly into the room above the people who may be in it. The overall disinfection of the room air is achieved by natural air circulation.

An overview of disinfection using such devices is shown in FIG. 1, which is taken from the ASHRAE Handbook-HVAC Applications (ASHRAE: American Society of Heating, Refrigerating, and Air-Conditioning Engineers, 2019, Handbook-HVAC Applications, Chapter 62.6. USA, Atlanta, GA.), with minor revisions. The diagram shows along the abscissa the room depth d_{horiz} (measured from the position of the device projected onto the floor, in units of 0.3048 m or feet) and along the ordinate the room height d_{horiz}, measured from the floor of the room, in units of 0.3048 m or feet. The conventional optical device 1000 is installed at a height of approximately 2.1 m. The reference signs 1010, 1020, 1030, 1040, and 1050 denote lines of equal local irradiance (1010:200 μW/cm², 1020:100 μW/cm², 1030:50 μW/cm², 1040:20 μW/cm², 1050:10 μW/cm²). The reference symbol 1060 indicates an area in which the irradiance is still above 0.2 μW/cm². In the active zone for disinfection, the irradiance should exceed a minimum value of 10 μW/cm² in order to be sufficiently effective. The so-called safe zone is located below the active zone. People should be able to stay here for longer (e.g. up to 8 hours). In this zone, the irradiance should generally be below 0.2 μW/cm². As can be seen, the UV-C radiation is essentially directed horizontally with a slight upward inclination so as not to affect the safe zone with UV-C radiation. Along the abscissa, the irradiance decreases considerably due to the increasing distance from the device. Disinfection can be achieved at a greater depth in the room by increasing the lamp output.

Due to the use of UV-C low-pressure lamps in conventional devices, which have a comparatively high etendue and consequently low radiance, it is necessary to use reflectors and slats to keep the radiation in the narrow zone under the ceiling indicated in FIG. 1. The louvres can absorb those radiation components that leave the reflectors of the device at larger angles (upwards or downwards relative to the horizontal plane). However, this in turn leads to efficiencies that are generally below 10 %. The resulting high power consumption with low long-distance effect and the highly uneven distribution of the irradiance in the area of the room ceiling therefore also have a negative impact on the cost-effectiveness of the devices—a higher number of devices per room ceiling area is required-and also on the complexity of the disinfection concept for a given room.

### SUMMARY

Embodiments provide improving the efficiency of the optical devices used for disinfection by UV radiation, in particular UV-C radiation, and at the same time providing greater protection for the people in the room or ensuring a sufficient safe zone.

The starting point is an optical device with a light source unit and a reflector. The optical device emits its radiation in the UV wavelength range, preferably in the UV-C wavelength range, in an essentially horizontal direction—with a slight inclination towards the ceiling—but in a significantly larger solid angle (at least when viewed vertically). Direct emission of ultraviolet light from the light source unit into the room is not intended. Instead, this task is performed by the reflector. For this purpose, the light source unit comprises a light-emitting surface that defines a light source plane and is configured to emit radiation in a UV wavelength range, in particular the UV-C wavelength range (e.g. 100 nm-280 nm). Alternatively, UV-A or UV-B radiation can also be used, particularly in conjunction with photocatalysis, if the ceiling is coated with titanium dioxide, for example. Aspects provide for a fairly large area to be irradiated, so that a good catalytic effect can potentially also be achieved in this case.

The light-emitting surface refers to the surface of the light source unit through which the light generated by it is emitted. It can, for example, be a light-emitting surface of an optical element, e.g. downstream of an LED etc., or directly an active surface of an LED if no other optical element is provided. The light-emitting surface lies in the light source plane and can span it. Furthermore, the light-emitting surface is arranged to emit the radiation in an angular range α, β, γ, δ relative to a main radiation direction Z that is perpendicular with respect to the defined light source plane. It is understood that because the light-emitting surface defines a plane, the angular range can be a maximum of 90°. When mounting the optical device in the room, the arrangement is preferably carried out in such a way that the main radiation direction Z coincides with the room height direction (e.g. the direction of the ordinate in FIG. 1). In this case, the light-emitting surface emits its maximum radiation into a half-space directed downwards towards the floor. According to a specific embodiment, the light source unit has a light source comprising one or more UV LEDs, in particular UV-C LEDs. The light-emitting surface could then, for example, be formed by the surface of the light sources. However, according to the embodiments to be described below, it is also possible to set up additional optical elements that interact with the light sources and then themselves provide the light-emitting surface of the light source unit.

The reflector is arranged at a predetermined distance from the light-emitting surface in the main radiation direction Z. It receives the radiation emitted by the light-emitting surface and reflects it as intended at least against the main radiation direction Z. This means that the reflected UV radiation has at least one directional component in the Z direction, which points back in the direction of the light source plane of the light-emitting surface.

The reflector also has a free-form shape with regard to the design of its reflective surface. The term free-form describes a technique with which freely definable optical effective surfaces can be manufactured very precisely. This technology is used in a wide variety of production processes. The term freeform stands for the technical possibility of producing reflective surfaces with an unconventional three-dimensional shape, but this does not rule out the possibility of finding an exact mathematical formulation for the geometric shape.

The freeform is designed in such a way that the radiation reflected by the reflector is projected onto a defined surface to be irradiated in the room. As the room itself is not part of the optical device, it can initially be a virtual surface that is in a given position relative to the reflector and the light source unit. This surface now extends against the main radiation direction Z-viewed from the reflector-beyond the light source plane. In the above-described case of mounting the optical device in the room near its ceiling, e.g. at a room height of approximately 2.1 m similar to that shown in FIG. 2, the light source plane is a horizontal plane perpendicular to the (vertical) Z direction, and the surface to be irradiated extends completely above it. The surface to be illuminated can preferably be the ceiling itself-or a limited part of it. The surface can be flat or curved or even stepped, but is in any case above the light source plane.

The free form of the reflector is now designed in such a way that the distribution of the irradiance of the radiation thrown onto the surface by the reflector is essentially homogeneous within the surface. This is by no means the expression of a mere wish for a result to be achieved. Rather, the inventor has applied a concept to the current problem in this respect which is derived from the publication Ries, H & Muschaweck, J.: “Tailored freeform optical surfaces” in J. Opt. Soc. Am. A/Vol. 19, No. 3/March 2002. It describes how freeform optical surfaces, which are embedded in three-dimensional space and need not exhibit symmetry, are designed to redistribute the radiation from a given very small light source onto a given reference surface in order to achieve a given irradiance distribution on this surface. The shape of the optical freeform surface is found by solving a series of partial non-linear differential equations. In most cases, there are only a few topologically different solutions if suitable boundary conditions are given. For the present, somewhat simpler case of a highly homogeneous distribution, it was found that, as a rule, only a single solution exists for the three-dimensional shape of the freeform.

In other words, the free form is practically clearly defined by the distance of the light-emitting surface from the reflector, by specifying a small light source unit and by specifying or defining the geometry and position of the surface to be irradiated in space relative to the light-emitting surface and the reflector and by specifying a homogeneous distribution of the irradiance, or in the exceptional case of more than one topological solution, at least obtainable from a selection of countable, very few solutions. The free form can be determined by computer and then produced.

The optical device can be used with particular advantage for disinfecting upper air layers in a room. In the case of a Lambertian radiator, the light-emitting surface radiates only into a half-space around the main radiation direction Z, which corresponds to the optical axis, preferably into an even narrower cone-shaped space around the main radiation direction Z. The free-form optical shape of the reflector reflects the radiation in a lateral direction onto a surface above the light source plane, which is defined by the essentially horizontal alignment of the light-emitting surface. The surface can be part of the ceiling. This is irradiated by the free-form surface with a homogeneous distribution of irradiance. This avoids local maxima, unlike in the past.

FIG. 1 also does not show the effect of a reflective ceiling. Depending on its positioning in FIG. 1, there is also a local maximum where the narrow active zone meets the ceiling. The consequence here would also be that the optical device would have to be installed at a greater height and may have to radiate more flatly in order to achieve a more even distribution on the surface of the room ceiling. However, this approach is limited by the absolute ceiling height. In contrast, embodiments of the invention allow the optical device to be applied even at low ceiling heights without interfering with the safe zone. This also improves cost-effectiveness.

As described, the use of the described light-emitting surface has the advantage that very small light source units can be realized. According to embodiments, this applies in particular to UV LEDs, preferably UV-C LEDs, which provide a high radiance or a low etendue. On the one hand, this makes it possible to calculate the free-form and use it at all. On the other hand, the power consumption is reduced with increased efficiency. Furthermore, the low etendue makes it possible to irradiate the reflector at extremely close range.

Due to the low source size and low etendue, it is now also possible to obtain a clearly defined area to be irradiated with homogeneous distribution of the irradiance. By positioning several optical devices appropriately relative to each other, the surfaces to be irradiated can also be placed adjacent to each other in accordance with the embodiments presented here, so that the entire surface of the ceiling is covered. The surfaces to be irradiated can be well delimited by the design of the free-form, i.e. where they are irradiated, the irradiance is homogeneous and outside, the irradiance drops to values that are negligible in comparison. In the conventional case (FIG. 1), on the other hand, there are usually no clear demarcations and it is therefore not possible to make any firm statements about the ranges of the respective optical devices.

According to a special further development of the optical device, the light source unit comprises one or more optical elements, each of which is assigned to the one or more UV LEDs or UV-C LEDs, whereby the light-emitting surface is formed by the one or more optical elements. With the aid of such special optical elements, for example, the angular range in which the light-emitting surface emits radiation relative to the main radiation direction Z can be further reduced.

For example, one or more optical elements can be formed by a light guide with an increasing cross-section from the light source in the form of a taper.

Alternatively, the one or more optical elements can comprise a lens to achieve the effect described.

Another alternative, for example, is for the optical element to comprise a light-absorbing ring that surrounds a central area with the light-emitting surface. The absorbing ring receives and absorbs radiation, which is emitted at large angles (relative to the main radiation direction). The geometry of the ring is not restricted. It can be a circular ring or a rectangular or square ring. Other shapes are also possible. Unlike the taper or lens proposed above, however, the power losses are greater and the efficiency lower due to absorption.

According to a further development of the optical device, the light-emitting surface has a width in a first direction X perpendicular to the main radiation direction Z and a length in a second direction Y perpendicular to the main radiation direction Z. The two directions X and Y are also perpendicular to each other. The two directions X and Y are also perpendicular to each other. Together they span the light source plane. The length is greater than the width.

In this aspect, the length of the light-emitting surface in the first direction X can be 16 mm or less as a maximum estimate, preferably 8 mm or less, more preferably 4 mm or less. Furthermore, the width of the light-emitting surface in the second direction Y can be 2 mm or less as a minimum estimate, preferably 1 mm or less, more preferably 0.5 mm or less.

During the investigations, it was found that the extent of the light-emitting surface in the direction transverse to the second direction Y can certainly be selected to be significantly larger, without the condition that the irradiance is homogeneously distributed in the surface to be irradiated having to be compromised. This makes it possible to apply significantly more radiation to the surface without significant losses.

Furthermore, according to embodiments of the optical device, an aspect ratio of the length to the width may be 32 or less, preferably 16 or less, more preferably 12 or less. Alternatively or additionally, the aspect ratio of the length to the width may be 2 or more, preferably 4 or more, more preferably 6 or more. These selection ranges have proven to be particularly efficient with regard to a balance between maintaining the condition (homogeneous distribution of the irradiance) and maintaining the etendue.

According to further embodiments, the one or more optical elements and the light-emitting surface may be arranged to emit radiation in a first angular range α, β relative to the main radiation direction Z within a plane spanned by the first direction X and the main radiation direction Z, wherein an angle of the first angular range α, β is at most 30° or more, preferably 45° or more, and 90° or less, preferably 85° or less. The extent of the homogeneity of the irradiance can be further improved by selecting the correspondingly reduced radiation angle in the X direction transverse to the plane spanned by the Y and Z directions, which at the same time can preferably represent the only symmetry plane of the projection system.

Alternatively or in addition to these embodiments, the one or more optical elements and the light emitting surface may be arranged to emit radiation in a second angular range α, β relative to the main radiation direction Z within a plane spanned by the second direction Y and the main radiation direction Z, wherein an angle of the second angular range α, β is at most 30° or more, preferably 45° or more, and 90° or less, preferably 70° or less. The same advantages as described above apply to this narrower selection range.

According to further embodiments, the predetermined distance between the light-emitting surface and the reflector in the main radiation direction Z can be a maximum of 20 mm or less, preferably 10 mm or less, and 2 mm or more, preferably 4 mm or more. These low values for the distance allow a particularly compact design.

According to aspects of the invention, the free-form shape of the reflector is such that the surface to be irradiated with homogeneous distribution of irradiance extends substantially in a second direction Y perpendicular to the main irradiation direction Z, starting from an axis parallel to the main irradiation direction, which also extends through the light-emitting surface. In other words, if the surface to be irradiated is, for example, a room ceiling (possibly including possible parts of the upper side walls), the free form leads to irradiation in a lateral direction and upwards towards the ceiling. Projected onto the ceiling, the optical device can therefore be located at the edge of the irradiated surface. This makes it possible, for example, to combine several optical devices in order to radiate in different directions from a common point in the room near the ceiling.

According to further embodiments, the surface to be irradiated extends essentially parallel to the light source plane. Then a ratio between the length of the surface to be irradiated in the second direction and a distance of the surface to be irradiated from the light source plane may be, for example, 15 or less, preferably 10 or less. Furthermore, the ratio can be 2.5 or more, preferably 5 or more. Alternatively or additionally, a ratio between the width of the surface to be irradiated in the first direction X and the distance of the surface to be irradiated from the light source plane may be 15 or less, preferably 10 or less, and 2.5 or more, preferably 5 or more.

Furthermore, as described, the free form of the reflector in the optical device can be mirror-symmetrical with respect to a plane which is spanned by the main radiation direction Z and the second direction X (i.e. the XY plane). The free form can, for example, have a height in the main radiation direction Z in a range from 15 mm to 90 mm, a width in the first direction X in a range from 40 mm to 180 mm, and a length in the second direction Y in a range from 50 mm to 250 mm.

Advantageously, the optical device can also have a blocking element that absorbs or reflects radiation which, due to its angle of radiation from the light-emitting surface, extends beyond one edge of the free form of the reflector. If necessary, the blocking element can extend around the entire edge of the free form. This prevents the radiation emitted from the light-emitting surface from entering the safe zone directly or reducing it.

### BRIEF DESCRIPTION OF THE DRAWINGS

Further advantages, features and details of the invention can be seen from the claims, the following description of preferred embodiments and from the drawings. In the figures, identical reference signs denote identical features and functions.

FIG. 1 shows in a diagram the effect of an optical device for disinfecting upper air layers in a room according to the state of the art;

FIG. 2 shows a schematic representation of an optical device according to an embodiment, which irradiates a surface of a ceiling above a safe zone;

FIG. 3 shows an optical device according to an embodiment with a light-emitting surface (a light source unit) and a reflector in side view (top), top view (bottom left) and front view (bottom right), with the coordinate axes defined in the directions X, Y and Z;

FIG. 4 shows a table with predetermined data for the area to be irradiated (length, width) in relation to the distance to the optical device and for the beam angle of the light-emitting surface and the distance between the light-emitting surface and the freeform in the Z direction, and with the resulting data for the freeform (length, width, height) in order to achieve a homogeneous irradiance over the area to be irradiated;

FIG. 5 shows a schematic representation of an angular range γ, δ of the radiation from the light-emitting surface in the YZ plane from FIG. 3;

FIG. 6 shows a schematic representation of an angular range α, β of the radiation from the light-emitting surface in the XZ plane from FIG. 3;

FIG. 7 shows a schematic view of an embodiment of the light-emitting surface extending in the XY plane with a rectangular ground plan that is basically elongated in the X direction;

FIG. 8 shows a schematic representation of a top view of a closed light source of the light source unit extending in the XY plane according to an embodiment;

FIG. 9 shows a schematic representation of a top view of a light source of the light source unit extending in the XY plane, which has a series of individual light sources, according to an alternative embodiment to FIG. 8;

FIG. 10 shows a schematic representation of a top view of a light source of the light source unit extending in the XY plane, which has a series of individual light sources separated from one another by a gap, according to an embodiment applied with respect to FIG. 9;

FIG. 11 shows a schematic representation of a top view of a light source of the light source unit extending in the XY plane, which has a two-row matrix of individual light sources, according to a further embodiment modified with respect to FIG. 9;

FIG. 12 shows a schematic representation of a side view (on the YZ plane) of an optical element arranged in front of the light source (as shown in one of FIGS. 8 to 11) and formed as a taper, which provides an opposite light-emitting surface, according to an embodiment;

FIG. 13 shows a schematic representation of a side view as in FIG. 12, but on the XZ plane;

FIG. 14 shows a schematic representation of a side view (e.g. on the YZ or XZ plane) of an optical element arranged in front of the light source and designed as a CPC, which is designed for a single light source in each case (as shown in FIGS. 9 to 11), according to an alternative embodiment to FIGS. 12 and 13;

FIG. 15 shows a schematic representation of a side view (e.g. on the XZ plane) of a plurality of optical elements according to FIG. 14, each arranged in front of the individual light sources of FIGS. 9 to 11 and designed as CPCs;

FIG. 16 shows a schematic representation of the beam path in the optical element or the respective optical elements from FIG. 14 or 15;

FIG. 17 shows a schematic representation of a side view (e.g. the YZ plane) of an optical element arranged in front of the light source and designed as a lens, which is designed for a single light source in each case (as shown in FIGS. 9 to 11), according to an alternative embodiment to FIGS. 12 to 16;

FIG. 18 shows a schematic representation of a side view (e.g. the YZ plane) of an optical element arranged in front of the light source and designed as an externally light-absorbing ring, which is designed for a single light source (as shown in FIGS. 9 to 11), according to an alternative embodiment to FIGS. 12 to 17;

FIG. 19 shows a schematic top view of the optical element from FIG. 18;

FIG. 20 shows a schematic representation of a cross-section in the YZ plane (symmetry plane) through the optical device with blocking element indicated on the outside according to a further embodiment;

FIG. 21 shows a schematic representation of the arrangement of the light-emitting surface and reflector relative to the surface to be illuminated on the ceiling in the room; and

FIG. 22 shows a top view of the area to be irradiated showing a largely homogeneous distribution of irradiance.

### DETAILED DESCRIPTION OF ILLUSTRATIVE EMBODIMENTS

In the following description of preferred embodiments, it should be noted that the present disclosure of the various aspects is not limited to the details of the structure and arrangement of the components as shown in the following description and in the figures. The embodiments can be put into practice or implemented in various ways. It should further be noted that the language and terminology used herein is for the purpose of specific description only and should not be construed as such in a limiting manner by those skilled in the art.

FIG. 2 shows a schematic representation of an optical device 10 according to an embodiment, which irradiates a surface 915 (with a predetermined length and width) of a ceiling 910 above a safe zone, which is represented by the dotted line 920 (or the line 920 represents its upper limit from a spatial point of view). To ensure that a person standing upright is safely captured in the safe zone, the height of line 920 should at least exceed its length. The optical device 10 emits its radiation 300 laterally in a selected angular range in an essentially horizontal direction with a component directed vertically upwards. Within the cone of radiation 300, the irradiance is sometimes more than 10 μW/cm² or 100 mW/m², which is to be regarded as a minimum condition for sufficient germicidal activity in the layers of air therein. Furthermore, the optical device 10 is configured in its structure and spatial arrangement in such a way that the surface 915 lying diagonally above it is irradiated with a homogeneous distribution of the irradiance there.

The ceiling 910 or the surface 915 to be irradiated can be idealized as a Lambertian radiator with a reflectivity R of 0.3. By way of example, the surface to be irradiated can be specified with a length and width of 5 m each. The radiation 950 reflected just as homogeneously from the ceiling 910 in this area should have an irradiance of only 0.2 μW/cm²=2 mW/m² or less in the safe zone so as not to cause any damage from UV radiation to the people in the safe zone. With these assumptions, a permissible upper limit of around 0.167 W can be determined for the power of the emitted radiation. In the simplified example, however, radiation contributions from any reflective walls are not taken into account. However, the example shows that an approach provided in accordance with the aspects and embodiments proposed here, in which the UV radiation 300 is directed through a reflector towards the ceiling from which it is reflected, on the one hand effectively disinfects a sufficient volume of air, and on the other hand does not lead to any impairment of the safe zone. Even low radiant powers can be used for this purpose and are sufficient. The low, purely exemplary estimated upper limits for the radiant power are compatible with the use of UV LEDs, and in particular available UV-C LEDs.

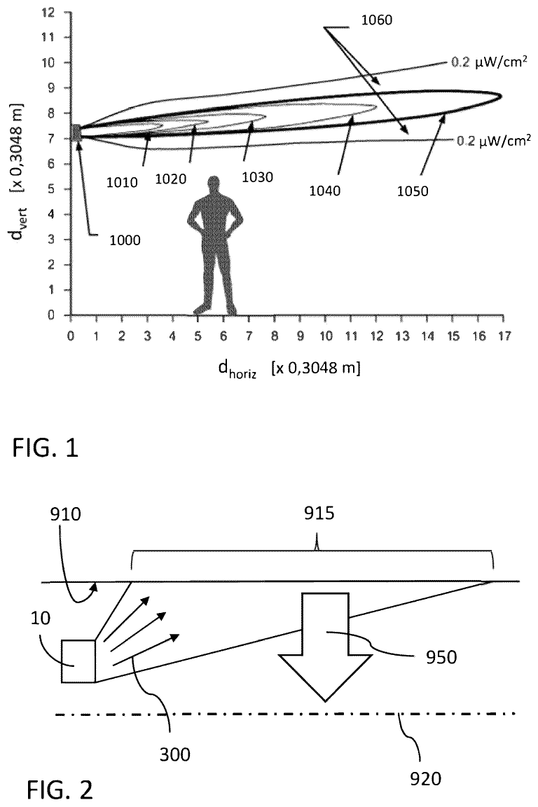

If the ceiling reflects the UV radiation (typically even as a Lambertian radiator with reflectivity R=0.3), this occurs with the same intensity across the surface. As a result, the safe zone is significantly less affected than in the conventional case (see FIG. 1). As described, the irradiance in the safe zone should not exceed 0.2 μW/cm². FIG. 1 shows that in the conventional device 1000 at a horizontal distance of about one meter (3 to 4 feet) from the position of the optical device, the corresponding area 1060 is shifted furthest downwards. The local maximum restricts the safe zone as a whole, or in other words: if this shift is too pronounced due to the local maximum, the optical device must be mounted even higher in the room to ensure a sufficient height for the safe zone.

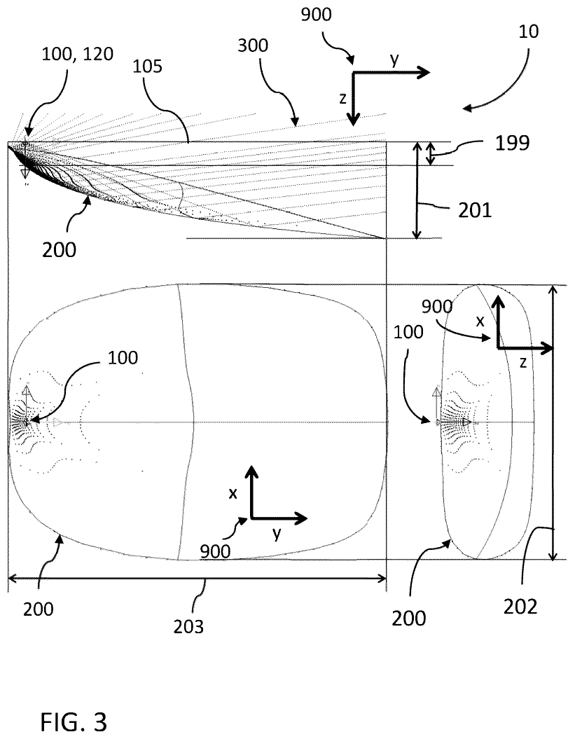

FIG. 3 shows an optical device 10 according to an embodiment that can be used, for example, in FIG. 2. The optical device comprises a light source unit 120, of which only a light-emitting surface 100 is shown, and a reflector 200. The upper part of FIG. 3 shows the side view, a top view is shown at the bottom left and a front view is shown at the bottom right. In this and all subsequent figures, the reference sign 900 denotes a Cartesian coordinate system with directions X, Y, Z. The Z direction is the vertical direction. The Z direction is the vertical direction in space and coincides with the main radiation direction Z of the light-emitting surface 100, which, however, points vertically downwards. The light-emitting surface 100 has a light source plane 105 that is perpendicular to the main radiation direction Z and is therefore spanned by a first direction X and a second direction Y.

Opposite the light-emitting surface 100 is a free-form reflector 200, which reflects the radiation 300 emitted by the light-emitting surface 100 according to its three-dimensional shape. The free form of the reflector 200 has a shell-like structure which opens towards the second direction Y and, according to this particular embodiment, occupies exactly one mirror or symmetry plane which extends in the YZ plane and in which the light-emitting surface 100 is also located. According to other embodiments, this mirror symmetry can be omitted.

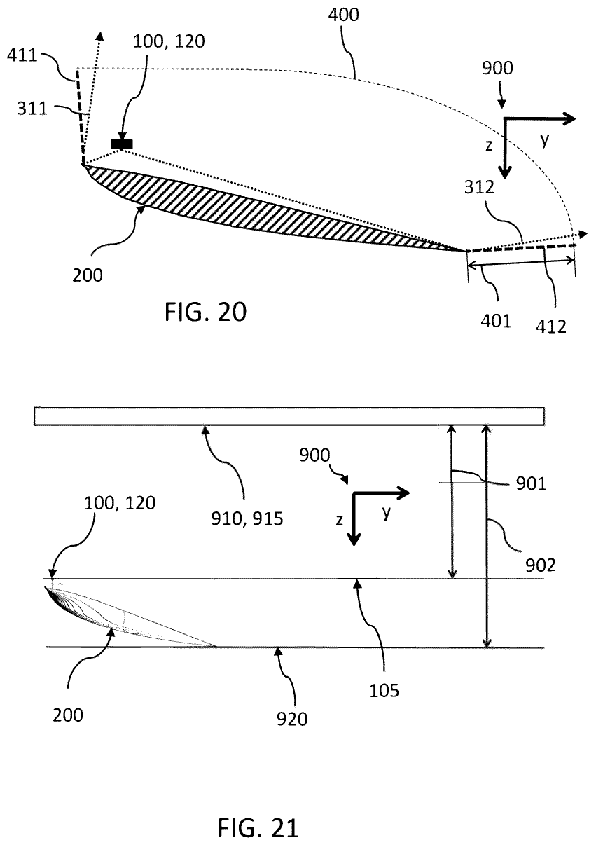

The shape and size of the free form of the reflector 200 is given by the requirement to achieve an optimum adaptation to a largely or even highly homogeneous irradiation of the surface 915 on the room ceiling 910 and at the same time the detection of as much as possible of the entire radiation 300 emanating from the light source plane 105. With reference to FIG. 20, measures are described below that are taken if not all of the radiation can be detected. The size of the freeform shape can be characterized by parameters such as a height 201 in the main radiation direction Z, a width 202 in the first direction X, and a length in the second direction Y. The concrete three-dimensional shape as such, on the other hand, cannot be indicated in simple notation and must generally be determined by programming, not only according to the embodiments shown here. For the corresponding method, which can also involve solving non-linear differential equations, reference is made to the publication Ries, H & Muschaweck, J.: “Tailored freeform optical surfaces” in J. Opt. Soc. Am. A/Vol. 19, No. 3/March 2002 and further references therein.

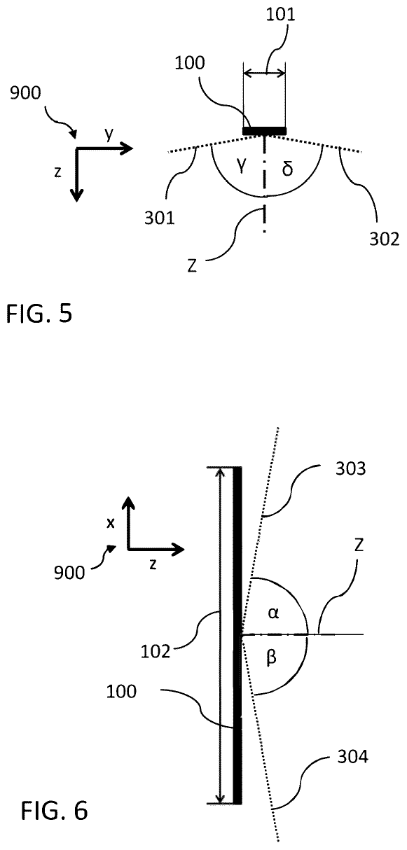

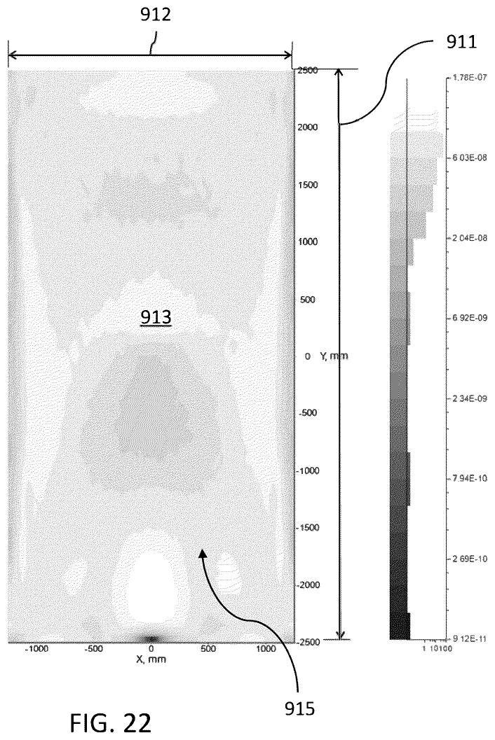

Certain specifications are made for the calculation of the free form, which ultimately clearly define the three-dimensional design as the result of the calculation. This includes defining the light source plane 105 and the plane of the surface to be illuminated (here: the ceiling 910) relative to each other (in the specific embodiments shown here, basically parallel to each other), their mutual distance 901 as well as the length 911 and the width 912 of the surface 915 to be illuminated (see FIG. 22), preferably normalized to the distance 901, and the position or orientation of the surface 915 within the plane of the ceiling 910, the distance 199 of the light-emitting surface 915 within the plane of the ceiling 910, and the distance 199 of the light-emitting surface 915 within the plane of the ceiling 910. orientation of the surface 915 within the plane of the ceiling 910, the distance 199 of the light-emitting surface 100 from the free-form surface of the reflector 200 along the main radiation direction Z, and the angular range α, β (in the XZ plane) or γ, δ (in the YZ plane) relative to the main radiation direction Z, in which the light-emitting surface 105 emits the radiation 300 in the direction of the free form (see FIGS. 5 and 6).

In the present embodiments, the surface 915 to be irradiated (as one of the specifications for calculating the free form) is oriented on the room ceiling 910 such that the comparatively small dimensioned light-emitting surface 100 falls on the edge of the surface 915 to be irradiated when projected along (or exactly against) the main radiation direction Z onto the plane of the room ceiling 910. This is not mandatory, but has the advantage that the surface 915 extends exactly from the optical device 10 into the room, which makes configuration in the room much easier for the user. Wall mounting is also possible.

By specifying the parameters mentioned or, in this case, special assumptions (planes parallel, optical device near or at the edge of the surface to be irradiated, and e.g. on its axis of symmetry, no radiation beyond the edge of the freeform), a clear solution for the freeform can now be determined as described, subject to the important requirement here that the irradiance resulting within the surface 915 is homogeneous.

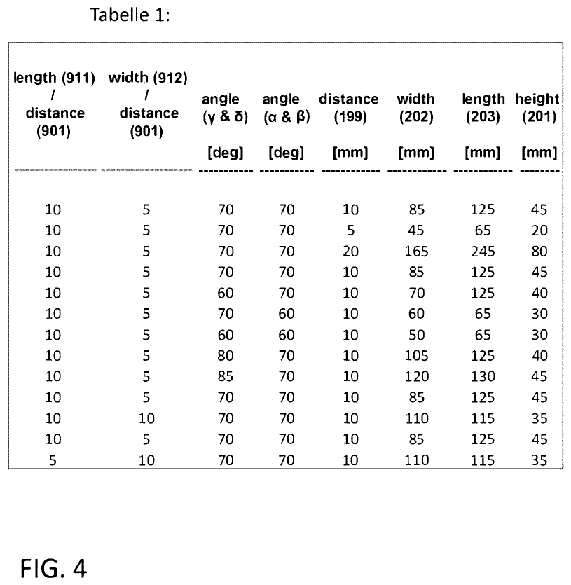

FIG. 4 shows a table 1 for this purpose, which reflects some of the specified parameters with selected values in columns 1 to 5. While the effect of the homogeneous radiation of the surface 915 can be meaningfully specified for the three-dimensional design of the free form, concrete values can be provided from the calculation at least for the height 201, the width 202 and the length 203, which are each clearly derived from the specified parameters. For 13 different constellations of length, width and spacing of the surface 915 as well as corresponding radiation angle ranges α, β (in the XZ plane) and γ, δ (in the YZ plane), these are given as examples in Table 1. Typical dimensions of the reflector 200 are listed in Table 1, but the actual dimensions can also deviate slightly by up to ±20% or more.

The optimum free form can therefore be calculated for the specified parameters. By varying the distance 901 from the ceiling 910, the surface 915 to be irradiated can be scaled proportionally with the length 911 and the width 912. This means that the free-form reflector 200 can be adapted to other target surfaces by changing the distance from the ceiling 910. The aspect ratio of the surface 915 to be irradiated can also be adapted by tilting it in the Z direction. Valid tilt angles should be less than 5°, preferably less than 2°.

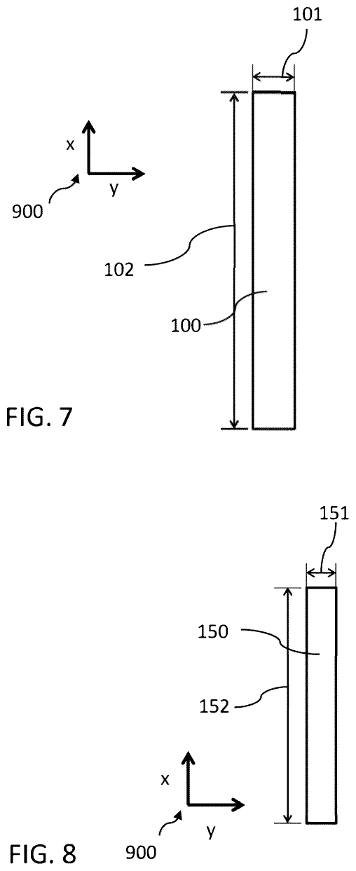

FIGS. 5 and 6 schematically illustrate the angle ranges γ, δ or α, β of the radiation from the light-emitting surface 100 in the YZ plane or the XZ plane from FIG. 3. In the special embodiments, the angles γ, δ on one side or α, β on the other side are identical to each other (see also Table 1, ideal cone), but can in principle also be selected differently. In FIGS. 5 and 6 and in particular in the top view of FIG. 7, the length 102 of the light-emitting surface 100 in the first direction X and the width 101 in the second direction Y are also shown. Optimal results of a homogeneous irradiance in the area 915 are achieved if the length 102 is not more than 4 mm and the width 101 is not more than 0.5 mm. The optimum aspect ratio is between 6 and 12 including these edge values. Optimum values for the maximum emission angle γ, δ relative to the main emission direction Z are in a range from 45° to 70° and for the maximum emission angle α, β relative to the main emission direction Z they are in a range from 45°to 85°. The reference signs 301, 302, 303 and 304 in FIGS. 5 and 6 denote the radiation emitted from the light-emitting surface 100 at the largest possible angle. The specified value ranges represent optimum results for the light-emitting surface 100. However, further value ranges are also specified in the preceding description, which also reflect even more preferred results for the optical device 10.

FIGS. 8 to 11 illustrate preferred embodiments for the light source 150 (associated with the light-emitting surface 100). FIG. 8 shows a schematic top view of a closed, contiguous light source 150 of the light source unit extending in the XY plane according to an embodiment. The light source 150 has a length 152 in the first direction X and a width 151 in the second direction Y. The aspect ratio corresponds approximately to that of the light-emitting surface 100. It should be noted that, if no further optical element is provided, the light source 150 itself can provide the light-emitting surface 100, which emits its radiation 300 directly onto the reflector 200. The light source in FIG. 8 can also be an elongated, single UV LED, in particular a UV-C LED.

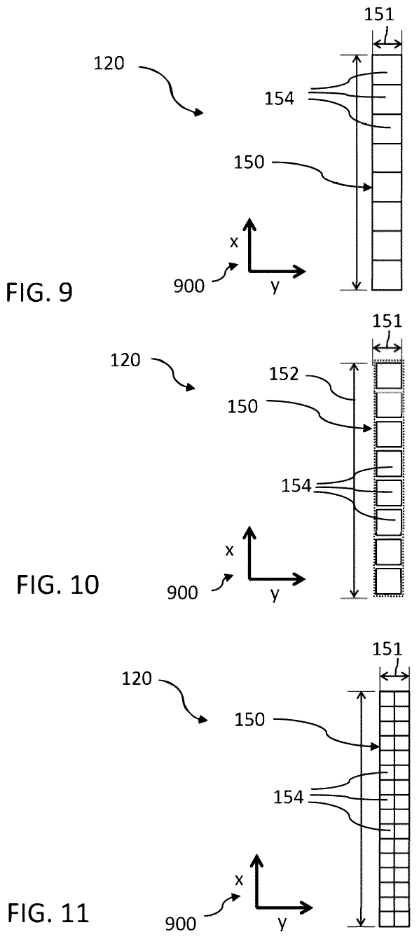

In FIG. 9, an alternative embodiment is shown in a top view of a light source 150 of the light source unit 120 extending in the XY plane, which, unlike in FIG. 8, has a series of directly adjacent individual light sources 154. These are preferably individual UV LEDs, in particular UV-C LEDs. With an optimum aspect ratio of between 6 and 12 and square LEDs, a row of 6 to 12 LEDs can therefore be considered. Without limiting the generality, purely exemplary UV-C LEDs with wavelengths of the light emitted by them in the range 260-280 nm, dimensions of the LED chips in the range 0.2×0.2 mm² to 1.0×1.0 mm² and outputs in the range 10 mW-150 mW come into consideration.

A modification of the embodiment shown in FIG. 9 is illustrated in FIG. 10. Here, the light source 150 of the light source unit 120, which also extends in the XY plane, has a series of individual light sources 154 separated from each other by a gap. This makes it possible to set up individual primary optics (optical elements 162, see in particular FIG. 15) for each of the individual light sources 154.

Furthermore, the idea on which FIGS. 9 and 10 are based can be applied to two-row or multi-row matrix-like arrangements of individual light sources 154 (UV LEDs or especially UV-C LEDs), as can be seen in FIG. 11. In these arrangements, it may even be possible to deviate from the simple rectangular shape of the light source 150 in plan view of the XY plane in order to enable advantageous geometric designs of the light-emitting surface 100.

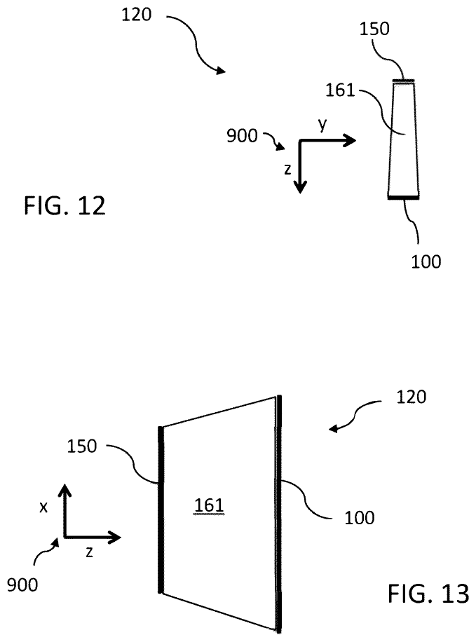

FIGS. 12 and 13 show an embodiment of the light source unit 120 with a light source 150 and an associated optical element 161, which is designed as a rod-shaped light guide (taper) that widens in cross-section. The light guide can be formed from solid material, in particular from a UV-resistant glass (quartz glass, sol-gel glass or cavity-coated aluminum). Other materials, possibly also UV-resistant plastics, can also be used. The cross-section in the XY plane is adapted to the geometry of the light source 150. The surface of the optical element 161 opposite the light source 150 can form the light-emitting surface. In principle, it is also conceivable that further optical elements are connected (combination of several optical elements).

The dimensions of the light-emitting surface 100 should be selected in relation to the emitter surface of the individual light sources 154 so that the emittance is maintained as far as possible. For LEDs, this generally means that the dimension xₑ of the emitter of the individual light source 154 and the dimension xₛ of the light-emitting surface 100 should behave as follows when viewed in the x-direction: xₑ≤xₛ−sin((α+β)/2), and analogously in the y-direction: yₑ≤yₛ−sin((γ+δ)/2). The narrower the beam angle, the larger the light-emitting surface 100 can be due to the widening taper (or the optical element 161).

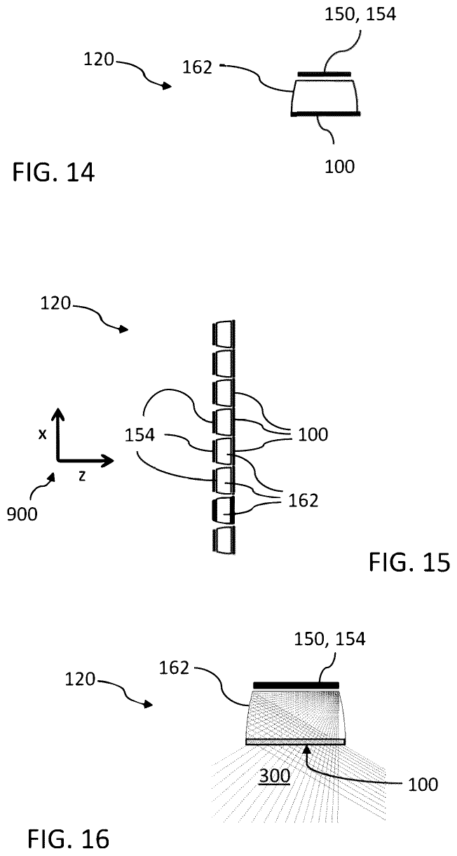

The optical element 161 of FIGS. 12 and 13, which serves as the primary optics, as well as corresponding optical elements 162, 163, 164 according to FIGS. 14 to 19 to be described below, adapt the radiation emitted by the light source 150 or the individual light sources 154 to form a common radiation source which can also be processed by the reflector 200 (radiation with a maximum angle α, β, γ, δ). the individual light sources 154 in order to form a common radiation source that can also be processed by the reflector 200 (radiation with maximum angle α, β, γ, δ and size of the light-emitting surface 100), so that—as described—the entire radiation is captured as far as possible and is emitted onto the surface 915 to be irradiated with a homogeneous distribution of irradiance.

FIGS. 14 to 16 show an alternative embodiment of a light source unit 120 to FIGS. 12 and 13. It is compatible with the rows of individual light sources 150 shown in FIGS. 9 to 11. Each of the individual light sources 150 is individually assigned its own optical element 162, which is designed as a CPC (compound parabolic concentrator, example of non-imaging optics). These optical elements 162, which are also designed as light guides or tunnels, each have a light entry surface facing the individual light source 150, which is opposite the light exit surface, as can best be seen in FIG. 16, which shows the path of the radiation 300 through the optical element 162.

The totality of these light-emitting surfaces forms the light-emitting surface 100. The light-emitting surface 100 therefore does not necessarily have to be contiguous. Preferably, however, the light-emitting surfaces form the light source plane 105. The path of the radiation 300 in FIG. 16 illustrates once again how the beam angle can be reduced compared to 90° (while maintaining the etendue) by enlarging the light-emitting surface compared to the light-emitting surface (by means of a taper).

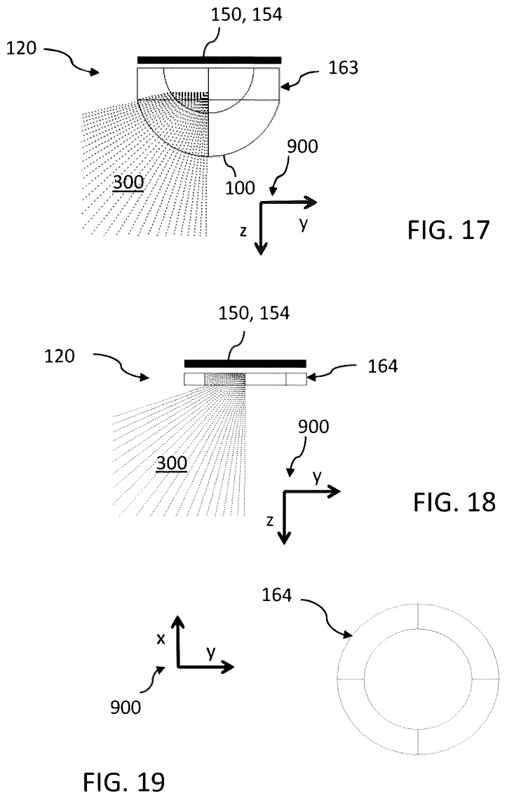

FIG. 17 shows a further embodiment of a light source unit 120. Here, an optical element 163 is provided opposite the light source 150 or each individual light source 154, which is designed as a lens. Lenses can be used in particular as individual primary optics for an array of emitters (individual light sources 150, UV LEDs, UV-C LEDs). The lenses can also be used to achieve a reduced beam angle, as can be seen from the path of the radiation in FIG. 17. The individual light emission surface is curved in this case. However, as a result of its arrangement, the surface made up of the large number of individual lenses forms a plane, which is referred to here as the light source plane 105. If only a single lens is present as optical element 163, the light source plane 105 is defined by a plane perpendicular to the corresponding optical axis, which is adjacent to the light-emitting surface of the lens.

Instead of the purely exemplary optical elements 161 and 162 presented above, a plurality of individual light-conducting rods can also serve as optical elements (not shown).

FIGS. 18 and 19 illustrate another embodiment of a light source unit 120 having an optical element 164 formed as a light absorbing ring formed, for example, of a metal such as aluminum or steel with an inner hole. Instead of having the property of absorbing light, the edge may also be reflectively coated towards the inner side, wherein the inner edge surface may be conical in shape to reduce the beam angle. The purpose of the absorbing ring is to adapt the angles of the outgoing radiation 300 to the requirements existing with respect to the light source plane 105 from the side of the reflector 200. It can also be an absorbing rectangular tube. The overall safety of the device can be improved by such absorbing elements by avoiding horizontally escaping radiation. As described, the optical elements 161, 162, 163 and/or 164 can also be combined, e.g. rods or lenses and absorbing rings.

FIG. 20 shows a cross-section through the optical device 10 with light source unit 120 and reflector 200. FIG. 20 shows an optional blocking element 400, which extends around the edge of the reflector 200. In the figure, the beams 311 and 312 indicate the edge of the radiation field with the greatest possible deflection during reflection. The blocking element 400 has sections 411 and 412 in cross-section, which ensure that larger deflection angles of these edge beams 311 and 312 do not go beyond a predetermined range, for example due to manufacturing tolerances or assembly errors, but are absorbed by the blocking element. The faint dash-dotted line connecting the sections 411 and 412 is purely schematic and not part of the cross-section: it is merely intended to illustrate that the blocking element 400 can surround the edge of the reflector 200 in a continuous manner.

FIG. 21 shows a purely exemplary arrangement of the optical device 10 with light source unit 120 and reflector 200 in the area of the room ceiling 910. The light source plane 105 defined by the light-emitting surface 100 extends horizontally in the room and parallel to the room ceiling 910. The light source plane 105 has a distance 901 to the room ceiling 910 or to the surface 915 to be irradiated, which is defined in advance therein. A lower edge of the reflector 200 forms a highest position for the safe zone in the room, designated in FIG. 21 by the horizontal surface or line 920. Since the room ceiling 910 is irradiated over a large area, the irradiance due to the reflected radiation 950 (see FIG. 2) does not decrease as quickly with increasing distance 902 from the room ceiling, at least in a room area below the surface 915 to be irradiated. However, due to the aspects and embodiments described, this is distributed so homogeneously that the irradiance values are sufficiently low and the safe zone (line 920) can already be defined close below the reflector.

FIG. 22 shows a top view of the result of a calculation of the free form shown in FIG. 3: for a projection onto the surface 915 to be irradiated with a length 911 in the Y direction of 5 m and a width 912 in the X direction of 2.5 m, a largely homogeneous distribution 913 of the irradiance can be seen. The calculation is also based on the fact that the device is located 0.5 m below the surface 915 at the position (x, y)=(0,-2500 mm). The light source 150 has the dimensions 4×0.5 mm. The distance 199 from the light source 150 to the reflector 200 is 10 mm. The light source 150 has an output of 1 W. On the right-hand side, a histogram of the values determined for all surface elements of the surface 915 is plotted in double-logarithmic representation. The values of the histogram are given in units of W/mm². The majority of the surface elements in area 915 have a converted irradiance of 3.51 to 10.4 μW/cm².

It should be noted that, according to a modification of the above embodiment, the ceiling 910 or the surface 915 to be irradiated can be coated with a photocatalytically active layer. This layer can comprise titanium dioxide, for example. In this case, the individual light sources 154 described above can also be designed as UV-A LEDs or UV-B LEDs that emit light in the corresponding wavelength ranges. Combinations of all the LED types mentioned are also conceivable. The UV light emitted by the optical device forms radicals on the surface of the titanium dioxide layer, for example, which are able to decompose organic substances, including germs or viruses, and oxidize gaseous substances.

The optical device 10 according to the embodiments shown above can have further components such as control electronics (not shown). The control electronics can include a dimmable LED control circuit. The dimming function allows the device to be set to an optimum germ-activating effect and at the same time achieve the requirements for the safety zone. As the optical properties of the room ceiling 910 and the walls are not the same everywhere, the driver current can be set by the control electronics so that the safety requirements are still achieved during installation.

Furthermore, the optical device 10 can have a cooling system (not shown). It makes sense to mount the LEDs on a heat dissipation line. The heat dissipation line can conduct the dissipated heat under or next to the reflector. This means that only a small amount of useful radiation is blocked by the heat dissipation line, any heat sinks or a corresponding fan.

In addition, optical device 10 can have an adjustment mechanism (not shown). This can be used to adjust the range (or the length 911 of the surface 915 to be irradiated in the Y direction) during installation or later during operation by tilting the optical device 10. This can be easily accomplished with standard components (joint, arm, housing, locking elements).

In addition, one or more fans for forced convection can be provided in combination with the optical device 10. In order to achieve an even better germicidal result, these fans can be installed at suitable positions so that an exchange between the air layers in the germicidal zone and the air layers below can be forced.

Furthermore, the optical device 10 can be extended by installing UV-C sensors or fluorescent materials that detect or visualize the UV-C radiation in the safe zone. For example, feedback can be provided in such a way that if the UV-C sensors detect an overshoot of a limit value in the safe zone, a signal is generated which the control electronics use to adjust the power (e.g. the drive current) with which the UV-C LEDs are supplied. An overshoot can, for example, result from additional reflection from side walls, or from an underestimated reflectivity of the ceiling 910 before installation, etc. On the other hand, the degradation of the UV-C LEDs over their service life can be taken into account by measuring via sensors by readjusting the output to values that are just compatible with the safe zone. A complete switch-off with an optional warning signal is also conceivable.

1-17. (canceled)

18. An optical device comprising:
a light source unit comprising a light source and a light-emitting surface, which defines a light source plane and is configured to emit radiation in a UV wavelength range, wherein the light-emitting surface is configured to emit the radiation from the light source unit in an angular range relative to a main radiation direction, which is perpendicular with respect to the light source plane;
a reflector, which is arranged at a predetermined distance from the light-emitting surface in the main radiation direction, and which is configured to receive the radiation emitted by the light-emitting surface and to reflect it at least in a direction opposite to the main radiation direction,
wherein the reflector comprises a free form, which is configured such that the radiation reflected by the reflector is projected onto a surface to be irradiated, which is defined in the room and which, in the direction opposite to the main radiation direction, extends beyond the light source plane as viewed from the reflector, and
wherein a distribution of an irradiance of the radiation projected onto the surface by the reflector is substantially homogeneous within the surface; and
a blocking element configured to absorb or reflect the radiation, which, due to its angle of radiation, passes from the light-emitting surface beyond an edge of the free form of the reflector.

19. The optical device according to claim 18, wherein the light source comprises one or more individual UV LEDs.

20. The optical device according to claim 19, wherein the light source unit comprises an optical element or a plurality of optical elements associated with the one or more UV LEDs, and wherein the light emitting surface comprises one or more optical elements.

21. The optical device according to claim 20, wherein the one or more optical elements are formed by an optical fiber that starts from the light source increases in cross-section in the manner of a taper.

22. The optical device according to claim 20, wherein the one or more optical elements comprise a lens.

23. The optical device according to claim 20, wherein the one optical element comprises a light-absorbing ring which encloses a central region with the light-emitting surface.

24. The optical device according to claim 18, wherein the light-emitting surface has a length in a first direction perpendicular to the main radiation direction and a width in a second direction perpendicular to the main radiation direction, which together span the light source plane, and wherein the length is greater than the width.

25. The optical device according to claim 24,
wherein the length of the light-emitting surface in the first direction is 16 mm or less, and/or
wherein the width of the light-emitting surface in the second direction is 2 mm or less.

26. The optical device according to claim 24,
wherein an aspect ratio of the length to the width is 32 or less, and
wherein an aspect ratio of the length to the width is 2 or more.

27. The optical device according to claim 24, wherein the one or more optical elements and the light emitting surface are configured to emit the radiation in a first angular range relative to the main radiation direction within a plane spanned by the first direction and the main radiation direction, and wherein a maximum angle of the first angular range is 30° or more, and 90° or less.

28. The optical device according to claim 24, wherein the one or more optical elements and the light-emitting surface are configured to emit the radiation in a second angular range relative to the main radiation direction within a plane spanned by the second direction and the main radiation direction, and wherein a maximum angle of the second angular range is 30° or more, and 90° or less.

29. The optical device according to claim 18, wherein the predetermined distance between the light-emitting surface and the reflector in the main radiation direction is 20 mm or less.

30. The optical device according to claim 18, wherein the free form of the reflector is configured such that the surface to be irradiated with homogeneous distribution of the irradiance extends substantially in a second direction perpendicular to the main radiation direction starting from an axis, which extends along the main radiation direction through the light-emitting surface.

31. The optical device according to claim 30,
wherein the surface to be irradiated extends substantially parallel to the light source plane, and
wherein a ratio between the length in the second direction and a distance of the surface to be irradiated from the light source plane is 15 or less, and/or
wherein a ratio between the width in the first direction and the distance of the surface to be irradiated from the light source plane is 15 or less.

32. The optical device according to claim 30, wherein the free form of the reflector is mirror symmetrical with respect to a plane spanned by the main radiation direction and the second direction, and has a height in the main radiation direction in a range from 15 mm to 90 mm, has a width in the first direction in a range from 40 mm to 180 mm, and has a length in the second direction in a range from 50 mm to 250 mm.

33. The optical device according to claim 18, wherein the light source comprises one or more individual light sources, which are configured to emit light in the UV-A wavelength range or in the UV-B wavelength range, and wherein the surface to be irradiated is coated with a layer which comprises titanium dioxide.
